(19) United States
(12) Patent Application Publication
(10) Pub. No.: US 2008/0026474 A1
(43) Pub. Date: Jan. 31, 2008

(54) ASSAY FOR DIFFERENTIATING COMPOUNDS THAT MODULATE THE EXTRINSIC AND/OR INTRINSIC COAGULATION PATHWAYS

(75) Inventors: Xinkang Wang, Berwyn, PA (US); Mei-Yin Hsu, Hillsborough, NJ (US); Pancras C. Wong, Plainsboro, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/782,660

(22) Filed: Jul. 25, 2007

Related U.S. Application Data
(60) Provisional application No. 60/833,674, filed on Jul. 27, 2006.

(51) Int. Cl. G01N 33/48

(52) U.S. Cl. 436/63

(57) ABSTRACT

Methods for differentiating compounds that modulate the extrinsic and/or intrinsic coagulation pathways are provided. Also provided are methods for identifying a compound that modulates the extrinsic coagulation pathway. In addition, methods for determining an effective dosage of an anticoagulant in a patient are provided.

### BACKGROUND OF THE INVENTION

The coagulation cascade consists of two separate initial pathways, the intrinsic and extrinsic pathways. The intrinsic and extrinsic pathways serve to activate the precursor protein prothrombin to the active enzyme thrombin. Thrombin is a multifunctional serine protease that plays a pivotal role in different biological phenomena, such as homeostasis, thrombosis, and cell differentiation. The intrinsic pathway of coagulation is also known as the “contact factor pathway”. Four proteins, namely, factor XII (FXII), prekeilikrein, high-molecular-weight-kininogen and C1-inhibitor, have been shown to be the major factors required for the activation and inhibition of the contact factor pathway. In the surface of the circulating system, mainly the cell membrane of blood and endothelial cells, the zymogens (factor XII and prekeilikrein) are converted by limited proteolysis into the active serine proteases. The activated FXII (FXIIa) activates factor XI and then the activated factor XI (FXIa) activates FIX of the intrinsic pathway.

The principal initiating pathway of in vivo blood coagulation following vascular injury is the extrinsic system. The critical component of the extrinsic pathway is tissue factor (TF). TF is the most potent trigger of the coagulation cascade and intravascular thrombus formation. When TF binds to factor VIIa (FVIIa), this complex activates both factor IX (FIX) and factor X (FX) to activated factor IXa (FIXa) and factor Xa (FXa), respectively. FXa catalyzes the generation of thrombin which subsequently activates platelets and cleaves fibrinogen to fibrin. Thrombin further facilitates its own generation by activating factor V (FV), factor VIII (FVIII) and FXI. Many experimental studies have demonstrated that inhibition of TF:FVIIa procoagulant activity is a powerful method to inhibit in vivo thrombosis.

Platelets support and maintain the coagulation process in different ways. Platelets provide the surface on which most of the thrombin required for clot formation is generated. One very important feature of platelets is their ability to adhere to sites of vascular injury and become activated. Upon activation, platelets undergo a number of changes that allow them to serve as a surface for the assembly and activation of tenase and prothrombinase complexes that generate Xa and thrombin, respectively. Therefore, TF can trigger platelet-rich plasma (PRP) to enter a potent reinforcement loop of thrombin generation, fibrin formation and platelet activation. In this setting, the mutual interaction of platelets and coagulation contribute to the physiological process of hemostasis.

While it is generally accepted that TF activation induces platelet aggregation, the effects on platelet function of compounds that modulate the extrinsic and/or intrinsic coagulation pathways have not been well characterized because platelet aggregation is typically studied under conditions that limit plasma coagulation. There is a need in the art for methods to differentiate compounds that modulate the extrinsic and/or intrinsic coagulation pathways. Furthermore, these methods may serve as tools to monitor the therapeutic effect of the compounds in patients.

### SUMMARY OF THE INVENTION

Some embodiments of the present invention are directed to a method for differentiating compounds that modulate the extrinsic and/or intrinsic coagulation pathways. The method involves a) collecting a blood sample in the presence of a calcium chelating agent and an inhibitor of contact activation, b) collecting a first and second platelet-rich plasma (PRP) sample from the blood sample in (a), c) adding a first test compound and a fibrin crosslinking inhibitor to said first PRP sample, d) adding a second test compound and a fibrin crosslinking inhibitor to said second PRP sample, e) incubating the mixtures from (c) and (d), f) inducing platelet aggregation in the mixtures from (c) and (d), g) measuring the time delay for said platelet aggregation in the mixtures from (c) and (d), and h) differentiating said first and second test compounds by comparing the time delay for platelet aggregation measured in (g) for said first and second PRP samples.

Other embodiments of the present invention are directed to a method for identifying a compound that modulates the extrinsic coagulation pathway. The method involves a) collecting a blood sample in the presence of a calcium chelating agent and an inhibitor of contact activation, b) collecting a PRP sample from the blood sample in (a), c) adding a test compound and a fibrin crosslinking inhibitor to said PRP sample, d) incubating the mixture from (c), e) inducing platelet aggregation, f) measuring the time delay for said platelet aggregation, and g) identifying a compound that modulates the extrinsic coagulation pathway by comparing the time delay for platelet aggregation measured in (f) to the time delay for platelet aggregation measured in a control PRP sample to which no test compound is added.

Some embodiments of the present invention are directed to a method for determining an effective dosage of an anticoagulant in a patient. The method involves a) collecting a first blood sample from a patient in the presence of a calcium chelating agent and an inhibitor of contact activation, b) collecting a first PRP sample from the blood sample in (a), c) adding a fibrin crosslinking inhibitor to said first PRP sample, d) administering an anticoagulant to said patient, e) collecting a second blood sample from said patient in the presence of a calcium chelating agent and an inhibitor of contact activation, f) collecting a second PRP sample from the blood sample in (e), g) adding a fibrin crosslinking inhibitor to said second PRP sample, h) inducing platelet aggregation in said first and second PRP samples, i) measuring the time delay for platelet aggregation, j) comparing the time delay for platelet aggregation in said first and second PRP samples, and k) increasing or decreasing the dosage of said anticoagulant based on the results in (j).

In some embodiments, the fibrin crosslinking inhibitor is pefabloc. In other embodiments, the inhibitor of contact activation is Corn Trypsin Inhibitor (CTI). In some embodiments, the calcium chelating agent is Na-Citrate or ethylenediamine tetraacetate (EDTA). In other embodiments, the PRP is human PRP. In some embodiments, the incubating is for at least 3 minutes at about 37° C.

In some embodiments, the concentration of tissue factor is from about 1-100 pM.

In other embodiments, the concentration of tissue factor is about 10 pM. In some embodiments, Innovin® is added as the tissue factor component.

In some embodiments, aggregation is induced by addition of tissue factor and Ca²⁺. In some embodiments, the Ca²⁺ is CaCl₂. In some embodiments, the concentration of Ca²⁺ is from about 5-10 mM. In other embodiments, the concentration of Ca²⁺ is about 7.5 mM.

In some embodiments, the time delay is measured under continuous stirring at 1000 rpm.

### BRIEF DESCRIPTION OF THE FIGURES

FIG. 1A illustrates the chemical structures of 2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid, 1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide, 3′-((2S,4R)-6-Carbamimidoyl-4-methyl-4-phenyl-1,2,3,4-tetrahydroquinolin-2-yl)-4-carbamoyl-5′-(3-methyl-butyrylamino)-biphenyl-2-carboxylic acid (chiral) and 1-[5-[(aminoiminomethyl)amino]-1-oxo-2-[[(1,2,3,4-tetrahydro-3-methyl-8-quinolinyl)sulfonyl]amino]pentyl]-4-methyl-2-piperidinecarboxylic acid (argatroban).

FIG. 1B illustrates the chemical structures of 6-[5-acetylamino-4,6-dihydroxy-2-(sulfooxymethyl)tetrahydropyran-3-yl]oxy-3-[5-(6-carboxy-4,5-dihydroxy-3-sulfooxy-tetrahydropyran-2-yl)oxy-6-(hydroxymethyl)-3-sulfoamino-4-sulfooxy-tetrahydropyran-2-yl]oxy-4-hydroxy-5-sulfooxy-tetrahydropyran-2-carboxylicacid (heparin), 1-[2-(2-tert-Butyl-phenoxy)-pyridin-3-yl]-3-(4-trifluoromethoxy-phenyl)-urea, 1-(2-aminomethylphenyl)-3-trifluoromethyl-N-[3-fluoro-2′-(aminosulfonyl)[1,1′-biphenyl)]-4-yl]-1H-pyrazole-5-carboxyamide and N6-methyl-2′deoxyadenosine-3′,5′-bisphosphate.

FIG. 2 shows representative tracings for the dose-dependent effect of FVIIa inhibitor, 2′-((6R,6aR, 11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid, on TF-induced platelet aggregation in human platelet-rich plasma. Vehicle, or 0.4, 1.26, 4.0, 12.6 or 25.2 μM (2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid) were used for the TF-induced platelet aggregation in human PRP. The arrow represents the point of time delay.

FIG. 3A illustrates the effects of FVIIa inhibitor (2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid) (Compound A) on time delay of TF-induced platelet aggregation in human platelet-rich plasma. 2×Base=1.25 μM.

FIG. 3B illustrates the effects of FXa inhibitor, (1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide) (Compound B), on time delay of TF-induced platelet aggregation in human platelet-rich plasma. 2×Base=0.4 μM.

FIG. 3C illustrates the effects of FXIa inhibitor, (3′-((2S,4R)-6-Carbamimidoyl-4-methyl-4-phenyl-1,2,3,4-tetrahydro-quinolin-2-yl)-4-carbamoyl-5′-(3-methyl-butyrylamino)-biphenyl-2-carboxylic acid) (Compound C), on time delay of TF-induced platelet aggregation in human platelet-rich plasma. 2×Base=26 μM.

FIG. 3D illustrates the effects of thrombin inhibitor, argatroban (Compound D), on time delay of TF-induced platelet aggregation in human platelet-rich plasma. 2×Base=0.62 μM.

FIG. 3E illustrates the effects of heparin (Compound E) on time delay of TF-induced platelet aggregation in human platelet-rich plasma. 2×Base=0.2 units.

FIG. 3F illustrates the effects of P2Y1 inhibitor, (1-[2-(2-tert-Butyl-phenoxy)-pyridin-3-yl]-3-(4-trifluoromethoxy-phenyl)-urea) (Compound F), on time delay of TF-induced platelet aggregation in human platelet-rich plasma. 2×Base=˜0 μM.

### DETAILED DESCRIPTION OF THE INVENTION

Thrombin generation and platelet activation are closely linked in the pathogenesis of thrombosis. Therefore, some embodiments of the present involve the use of time delay for TF-induced platelet aggregation to differentiate various compounds that modulate the extrinsic and/or intrinsic coagulation pathways.

An embodiment of the present invention is a method for differentiating compounds that modulate the extrinsic and/or intrinsic coagulation pathways. The method involves a) collecting a blood sample in the presence of a calcium chelating agent and an inhibitor of contact activation, b) collecting a first and second platelet-rich plasma (PRP) sample from the blood sample in (a), c) adding a first test compound and a fibrin crosslinking inhibitor to said first PRP sample, d) adding a second test compound and a fibrin crosslinking inhibitor to said second PRP sample, e) incubating the mixtures from (c) and (d), f) inducing platelet aggregation in the mixtures from (c) and (d), g) measuring the time delay for said platelet aggregation in the mixtures from (c) and (d), and h) differentiating said first and second test compounds by comparing the time delay for platelet aggregation measured in (g) for said first and second PRP samples.

Another embodiment of the present invention is a method for identifying a compound that modulates the extrinsic coagulation pathway. The method involves a) collecting a blood sample in the presence of a calcium chelating agent and an inhibitor of contact activation, b) collecting a PRP sample from the blood sample in (a), c) adding a test compound and a fibrin crosslinking inhibitor to said PRP sample, d) incubating the mixture from (c), e) inducing platelet aggregation, f) measuring the time delay for said platelet aggregation, and g) identifying a compound that modulates the extrinsic coagulation pathway by comparing the time delay for platelet aggregation measured in (f) to the time delay for platelet aggregation measured in a control PRP sample to which no test compound is added.

A further embodiment of the present invention is a method for determining an effective dosage of an anticoagulant in a patient. The method involves a) collecting a first blood sample from a patient in the presence of a calcium chelating agent and an inhibitor of contact activation, b) collecting a first PRP sample from the blood sample in (a), c) adding a fibrin crosslinking inhibitor to said first PRP sample, d) administering an anticoagulant to said patient, e) collecting a second blood sample from said patient in the presence of a calcium chelating agent and an inhibitor of contact activation, f) collecting a second PRP sample from the blood sample in (e), g) adding a fibrin crosslinking inhibitor to said second PRP sample, hi) inducing platelet aggregation in said first and second PRP samples, i) measuring the time delay for platelet aggregation, j) comparing the time delay for platelet aggregation in said first and second PRP samples, and k) increasing or decreasing the dosage of said anticoagulant based on the results in (j).

In some embodiments, the blood sample is collected in the presence of a calcium chelating agent and an inhibitor of contact activation. For example, the blood sample may be collected into a container comprising a calcium chelating agent and an inhibitor of contact activation. The blood sample may be collected from any mammal. Exemplary mammals include, but are not limited to, mice, cows, dogs and humans. In some embodiments, the mammal is a human.

The calcium chelating agent may be any agent that is capable of chelating calcium. Exemplary calcium chelating agents comprise, but are not limited to, Na-Citrate and EDTA.

The inhibitor of contact activation may be any compound that is capable of inhibiting contact activation. Exemplary inhibitors of contact activation comprise, but are not limited to, corn trypsin inhibitor, FXIIa inhibitors and kallikrein inhibitors.

The PRP sample is collected from a blood sample. Methods for collecting a PRP sample from a blood sample are well known in the art. In some embodiments, the PRP sample is collected from a blood sample by centrifugation.

The fibrin crosslinking inhibitor may be any compound that is capable of inhibiting fibrin crosslinking. Exemplary fibrin crosslinking inhibitors comprise, but are not limited to, prefabloc. The purpose of the fibrin crosslinking inhibitor is to prevent the final step of coagulation, i.e., fibrin crosslinking for thrombus formation, and to enhance platelet aggregation.

The test compound may be any compound. In some embodiments, the test compound is a compound that modulates the extrinsic and/or intrinsic coagulation pathways. As used herein, the term “modulates” refers to the ability of a compound to alter the function of the extrinsic and/or intrinsic coagulation pathways. The alteration may be enhancement, diminishment, activation and/or inactivation of the coagulation pathways. In some embodiments, the test compound is an anticoagulant. In other embodiments, the test compound may be a FVIIa inhibitor, a FXa inhibitor, a thrombin inhibitor, heparin, an FXIa inhibitor, a P2Y1 inhibitor or a P2Y12 inhibitor. Exemplary test compounds comprise, but are not limited to 2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid, 1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide, 1-(2-aminomethylphenyl)-3-trifluoromethyl-N-[3-fluoro-2′-(aminosulfonyl)[1,1-biphenyl)]-4-yl]-1H-pyrazole-5-carboxyamide, 3′-((2S,4R)-6-Carbamimidoyl-4-methyl-4-phenyl-1,2,3,4-tetrahydro-quinolin-2-yl)-4-carbamoyl-5′-(3-methyl-butyrylamino)-biphenyl-2-carboxylic acid, 1-[2-(2-tert-Butyl-phenoxy)-pyridin-3-yl]-3-(4-trifluoromethoxy-phenyl)-urea, N6-methyl-2′-deoxyadenosine-3′,5′-bisphosphate, argatroban and heparin.

The test compounds of the present invention may be useful for treating thromboembolic disorders. The term “thromboembolic disorders” as used herein includes, but is not limited to, specific disorders selected from unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary and cerebral arterial thrombosis, cerebral embolism, kidney embolisms, pulmonary embolisms, and thrombosis resulting from medical implants, devices, or procedures in which blood is exposed to an artificial surface that promotes thrombosis. The medical implants or devices include, but are not limited to: prosthetic valves, indwelling catheters, stents, and vessel grafts. The procedures include, but are not limited to: cardiopulmonary bypass and hemodialysis. It is noted that thrombosis includes occlusion (e.g. after a bypass) and reocclusion (e.g., during or after percutaneous transluminal coronary angioplasty). The term “stroke”, as used herein, refers to embolic stroke or atherothrombotic stroke arising from occlusive thrombosis in the carotid communis, carotid internal or intracerebral arteries.

In some embodiments, the mixture is incubated. The mixture is incubated at a temperature and for a time period that is sufficient to allow the compounds bind to their selective molecular target(s). Incubation temperatures and time periods may be determined using techniques that are well known to individuals of skill in the art. Exemplary incubation temperatures and time periods comprise, but are not limited to, about 37° C. and about 3 minutes.

In some embodiments, platelet aggregation is induced in the mixture. Platelet aggregation may be induced by any method that causes platelets to aggregate. For example, in some embodiments, platelet aggregation is induced by addition of tissue factor and calcium (Ca²+), ADP, collagen, thrombin, thrombin receptor activating peptide (TRAP), arachidonic acid and tissue factor.

The tissue factor can be a partially purified preparation or a purified recombinant mammalian tissue factor. In some embodiments, the tissue factor may be added as a single ingredient. In other embodiments, the tissue factor may be added in the form of a tissue factor preparation. The tissue factor preparation may contain ingredients such as lipds and salts. In some embodiments, the tissue factor preparation is Innovin® (Dade Behring, Deerfield, Ill.), which contains a human recombinant form of lipated tissue factor. In some embodiments, the concentration of tissue factor is about 1-100 pM. In other embodiments, the concentration of tissue factor is about 10 pM.

The Ca²⁺ may be any Ca²⁺- containing compound, such as CaCl₂. In some embodiments, the concentration of Ca²⁺ is about 5-10 mM. In other embodiments, the concentration of Ca²⁺ is about 7.5 mM.

The time delay for platelet aggregation may be measured by any method that allows for measurement of platelet aggregation. Methods for measuring platelet aggregation are well known to those of skill in the art. Exemplary methods for measuring platelet aggregation include, but are not limited to use of a PA-200 platelet aggregation analyzer or a Chrono-Log aggregometer. In some embodiments, platelet aggregation is induced under continuous stirring at 1000 rpm in a PA-200 platelet aggregation analyzer.

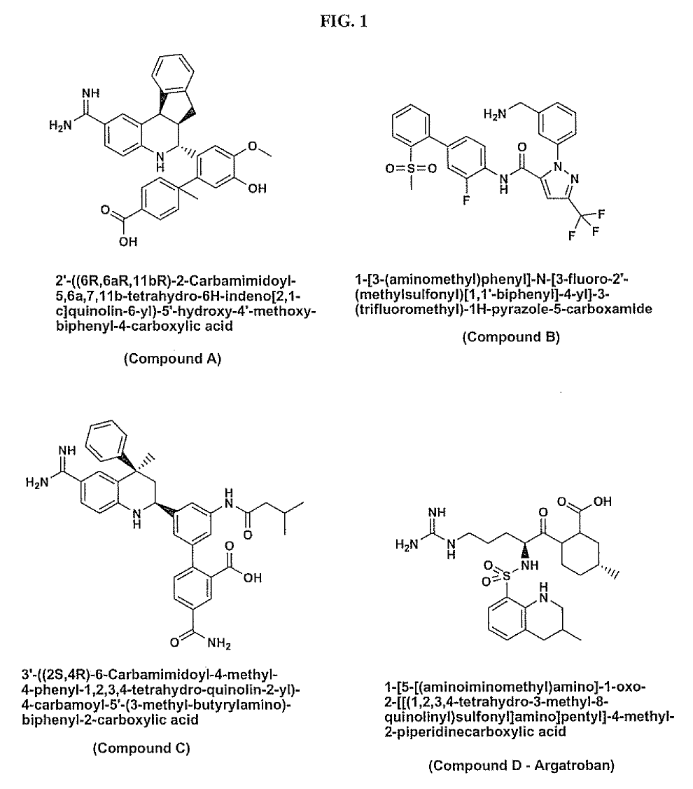

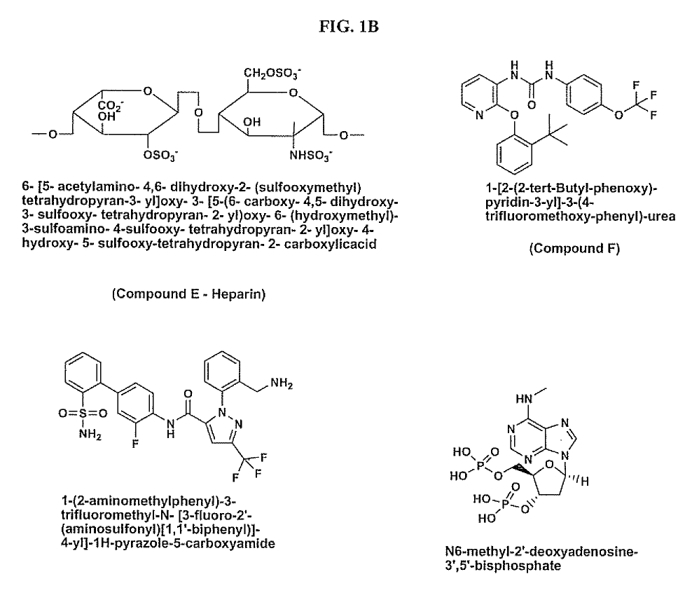

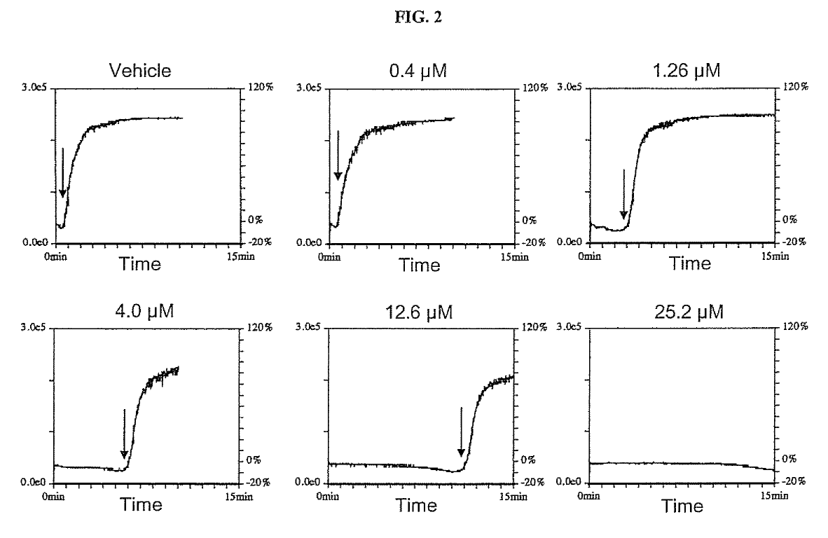

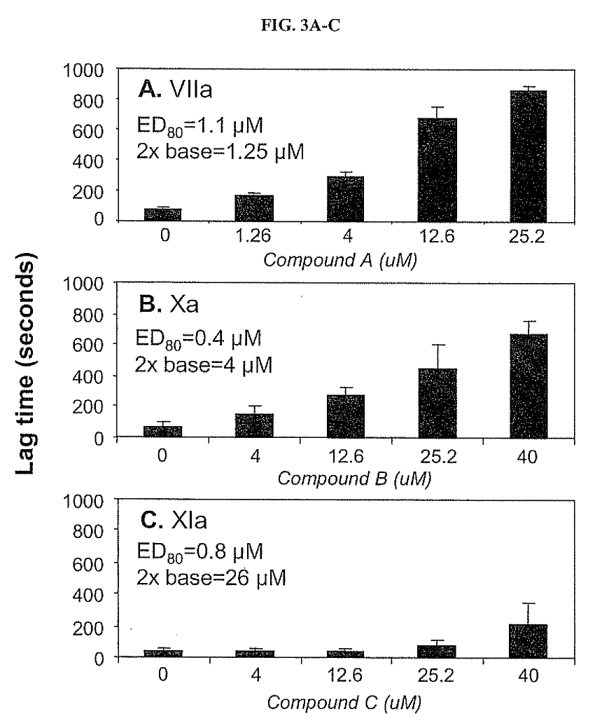

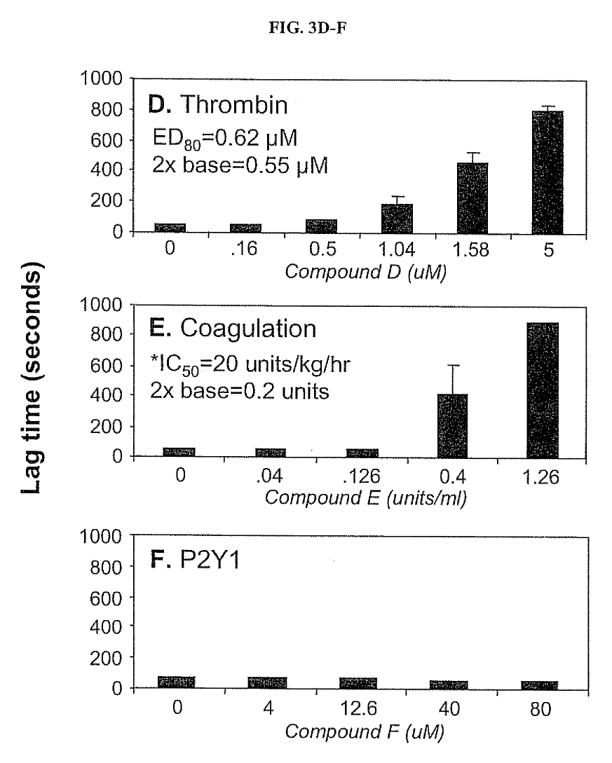

Some embodiments of the present invention involve methods for differentiating compounds that modulate the extrinsic and/or intrinsic coagulation pathways. Compounds may be differentiated by comparing the time delay for platelet aggregation. As described in the example, dose-dependent effects for the time delay for platelet aggregation were measured for various compounds. The effects are illustrated in FIGS. 2 and 3. These results demonstrate that the time delay for TF-induced platelet aggregation is a parameter that differentiates various anticoagulants targeting the coagulation cascade. Because the interaction of TF with FVIIa represents the first step in the extrinsic coagulation cascade, FVIIa inhibitors should be extremely efficacious in affecting the time delay for TF-induced platelet aggregation. The 2×Base value (2×basal levels), which represents concentrations required to double the time for TF-induced platelet aggregation, of FVIIa inhibitor 2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid (see FIG. 3A) in this assay is very similar to the ED₈₀ value of this FVIIa inhibitor in the rabbit ECAT model. Downstream antagonists, such as FXa inhibitors 1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide (see FIG. 3B) and 1-(2-aminomethylphenyl)-3-trifluoromethyl-N-[3-fluoro-2′-(aminosulfonyl)[1,1′-biphenyl)]-4-yl]-1H-pyrazole-5-carboxyamide (data not shown), required about 10-fold the 2×Base concentration in order to achieve an ED₈₀ value in the rabbit ECAT model. On the other hand, since FXIa is in the upstream of the intrinsic pathway, the FXIa antagonist barely showed any effect on TF-induced platelet aggregation (see FIG. 3C). The weak efficacy detected at the high dose of FXIa antagonist (40 μM 3′-((2S,4R)-6-Carbamimidoyl-4-methyl-4-phenyl-1,2,3,4-tetrahydro-quinolin-2-yl)-4-carbamoyl-5′-(3-methyl-butyrylamino)-biphenyl-2-carboxylic acid) might reflect its cross reactivity to FVIIa, or the limited involvement of FXIa in the feedback and amplification of thrombin in the coagulation cascade.

Potent effects on the time delay of TF-induced platelet aggregation were observed for argatroban and heparin (see FIGS. 3D and 3E). The efficacious effect of argatroban may be explained by the fact that platelet activation may be triggered by even a trace amount of pre-existing thrombin in the PRP. The potent role of heparin might reflect its ability to block multiple proteases in the coagulation cascade, including FVIIa.

Based upon their in vitro (2×Base) and in vivo (EC₈₀) effects the antithrombotic drugs tested can be rank-ordered in overall efficacy as follows: FVIIa inhibitor=thrombin inhibitor>FXa inhibitor>>FXIa inhibitor>P2Y1 inhibitor=P2Y12 inhibitor.

Other embodiments of the present invention involve methods for identifying a compound that modulates the extrinsic coagulation pathway. A compound that modulates the extrinsic coagulation pathway may be identified by comparing the time delay for platelet aggregation in a PRP reaction mixture containing the test compound to the time delay for platelet aggregation in a control PRP reaction mixture to which no test compound is added. A difference in time delay for platelet aggregation compared to the control PRP reaction mixtures indicates that the test compound modulates the extrinsic coagulation pathway. For example, FVIIa inhibitor, 2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid, showed a concentration-dependent effect on the time delay for platelet aggregation (FIG. 3A). A longer time delay for platelet aggregation indicates the presence of a higher concentration of this inhibitor in the PRP. In contrast, a shorter time delay for platelet aggregation indicates the presence of a lower concentration of this inhibitor.

Some embodiments of the present invention involve methods for determining all effective dosage of an anticoagulant in a patient. The anticoagulant may be any anticoagulant including, but not limited to, a factor VIIa inhibitor. The patient may be suffering from a disease or disorder including, but not limited to thromboembolic disorders. The term “thromboembolic disorders” as used herein includes, but is not limited to, specific disorders selected from unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary and cerebral arterial thrombosis, cerebral embolism, kidney embolisms, pulmonary embolisms, and thrombosis resulting from medical implants, devices, or procedures in which blood is exposed to an artificial surface that promotes thrombosis. The medical implants or devices include, but are not limited to: prosthetic valves, indwelling catheters, stents, and vessel grafts. The procedures include, but are not limited to: cardiopulmonary bypass and hemodialysis. It is noted that thrombosis includes occlusion (e.g. after a bypass) and reocclusion (e.g., during or after percutaneous transluminal coronary angioplasty). The term “stroke”, as used herein, refers to embolic stroke or atherothrombotic stroke arising from occlusive thrombosis in the carotid communis, carotid internal or intracerebral arteries.

In some embodiments, a first blood sample is collected from the patient prior to administration of an anticoagulant. A second blood sample may be collected from the patient at any time after administration of an anticoagulant. Determining the period of time between administration of the anticoagulant to the patient and collection of a second blood sample from the patient is within the skill of an ordinary artisan. Exemplary time periods are 1, 5, 10, 12, 24 and 48 hours after administration of an anticoagulant. Additional blood samples may collected from the patient. In some embodiments, a third, fourth or fifth blood sample may be collected from the patient at time periods that are easily determined by an ordinary artisan.

A comparison of the time delay for platelet aggregation in said first and subsequent PRP samples indicates whether it is desirable to increase or decrease the dosage of anticoagulant to the patient. For example, if the time delay for platelet aggregation decreases, a medical practitioner may administer a larger dose of anticoagulant to the patient. On other hand, if the time delay for platelet aggregation increases, a medical practitioner may reduce the dosage of anticoagulant.

Some embodiments of the present invention provide articles of manufacture, such as kits and packages for differentiating compounds that modulate the extrinsic and/or intrinsic coagulation pathways, identifying a compound that modulates the extrinsic coagulation pathway and determining an effective dosage of an anticoagulant. In one embodiment, the present invention provides a kit including Na-Citrate, prefabloc, corn trypsin inhibitor, CaCl₂ and tissue factor for use in differentiating compounds that modulate the extrinsic and/or intrinsic coagulation pathways, identifying a compound that modulates the extrinsic coagulation pathway or determining an effective dosage of an anticoagulant

The methods of the present invention can be performed manually or performed using an automated system to achieve high-throughput. Techniques for performing high-throughput assays include use of microtiter plates or pico-, nano- or micro-liter arrays. The assays of the invention are designed to permit high throughput screening of large compound libraries, e.g., by automating the assay steps and providing candidate factor VIIa inhibitors from any source to assay. Assays which are run in parallel on a solid support (e.g., microtiter formats on microtiter plates in robotic assays) are well known. Automated systems and methods for detecting and measuring changes in optical detection (or signal) are known. Furthermore, in the assays of the invention, it is desirable to ran positive controls to ensure that the components of the assays are working properly.

### EXAMPLE

The invention is illustrated by the following example. The example is illustrative only and does not limit the scope of the invention in any way.

### Materials and Methods

### Reagents:

Corn Trypsin Inhibitor (Cat# CTI-01), an inhibitor of FXII activation, was purchased from Haematologic Technologies Inc. Pefabloc (Cat# 09911-1G-B), inhibitor of fibrin crosslinking, was from Pentapharm. Innovin® (Cat# 84212-50), a human recombinant form of lipated tissue factor, was from Dade Behring. Selective small molecular inhibitors for FVIIa (2′-((6R,6aR, 11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid), FXa (1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide and 1-(2-aminomethylphenyl)-3-trifluoromethyl-N-[3-fluoro-2′-(aminosulfonyl)[1,1′-biphenyl)]-4-yl]-1H-pyrazole-5-carboxyamide), FXIa (3′-((2S,4R)-6-Carbamimidoyl-4-methyl-4-phenyl-1,2,3,4-tetrahydro-quinolin-2-yl)-4-carbamoyl-5′-(3-methyl-butyrylamino)-biphenyl-2-carboxylic acid) and P2Y1 (1-[2-(2-tert-Butyl-phenoxy)-pyridin-3-yl]-3-(4-trifluoromethoxy-phenyl)-urea) were synthesized. Aspirin was purchased from Sigma. Argatroban was purchased from GSK (NDC0007-4407-01 Rx, 100 mg/ml). Heparin was purchased from Upjohn (NDC0009-0268-01, 1000 units/ml).

### Blood Sampling:

Nine parts of human blood were collected into one part of 3.8% Na-Citrate containing 50 μg/ml cone trypsin inhibitor (CTI) and then centrifuged at 150×g for 10 min to obtained platelet-rich plasma (PRP). The remaining samples were then centrifuged at 300×g for 10 min to obtain platelet-poor plasma (PPP). Platelet count was adjusted to 2-2.5×10⁸ cells/ml with platelet-poor plasma.

### Platelet Aggregation Assay:

PRP (275 μl) sample was briefly mixed with 25 μl of 36 mg/ml pefabloc. Various compounds (in a volume of 1.2 μl) were added and incubated at 37° C. for 3 min in a cylindrical glass cuvette. Platelet aggregation was induced by 4 μl of Innovin®-CaCl₂ mixture and measured under continuous stirring at 1000 rpm in a PA-200 platelet aggregation analyzer (Kowa, Tokyo, Japan). Platelet-poor plasma was used as a reference. Platelet aggregation curves defined by changes in light transmission over time were recorded for 15 min. The lag time was defined as the time from CaCl₂ addition until the light transmittance reached 1%. The concentration of Innovin® and CaCl₂ was adjusted for each sample so that the average control lag time was from 50 to 70 seconds.

### Rabbit ECAT Model:

The rabbit ECAT model used in the present study was as described previously (Wong et al., 2002). Briefly, male, New Zealand, white rabbits were anesthetized with ketamine (50 mg/kg+50 mg/kg/h i.m.) and xylazine (10 mg/kg+10 mg/kg/h i.m.). These anesthetics were supplemented as needed. An electromagnetic flow probe was placed on a segment of an isolated carotid artery to monitor blood flow. Thrombus formation was induced by electrical stimulation of the carotid artery for 3 min at 4 mA using an external stainless steel bipolar electrode. 2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid, 1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide, 3′-((2S,4R)-6-Carbamimidoyl-4-methyl-4-phenyl-1,2,3,4-tetrahydro-quinolin-2-yl)-4-carbamoyl-5-(3-methyl-butyrylamino)-biphenyl-2-carboxylic acid, argatroban, or vehicle (6 ml/kg/h) was infused intravenously 1 h before the electrical stimulation of the carotid artery and continuously during the 90-min period of thrombus formation. Plasma concentrations were determined in blood samples taken at the end of the experiment by liquid chromatography-tandem mass spectrometry.

Carotid blood flow was measured continuously over the 90-min period of thrombus formation and total carotid blood flow was calculated as an area under the carotid blood flow-time curve. Preservation of total CBF relative to sustained baseline flow was used to assess antithrombotic activity. The drug concentrations which maintained total blood flow at 80% of baseline (ED₈₀) was the common comparator among antithrombotic agents.

### Rabbit AV Shunt Model:

The rabbit AV shunt model was as described previously (Wong et al. J Pharmacol Exp Ther. 2000;292-351-357.). Briefly, male New Zealand White rabbits that were anesthetized with ketamine (50 mg/kg i.m.) and xylazine (10 mg/kg i.m.) were used. The femoral artery, jugular vein, and femoral vein were isolated and catheterized. A saline-filled arteriovenous (AV) shunt device was connected between the femoral arterial and femoral venous cannulas. The AV shunt device consisted of an outer piece of Tygon tubing (length, 8 cm; i.d., 7.9 mm) and an inner piece of tubing (length, 2.5 cm; i.d., 4.8 mm). The AV shunt also contained an 8-cm length of 2-0 silk thread (Ethicon, Somerville, N.J.). Blood flowed from the femoral artery via the AV shunt into the femoral vein. The exposure of flowing blood to a silk thread induced the formation of a significant thrombus. After 40 min, the shunt was disconnected and the silk thread, which was covered with thrombus, was weighed.

Various doses of heparin (units/kg/hr) or vehicle (saline) was given as continuous i.v. infusion via the jugular vein, starting 1 h before blood was circulated in the shunt and continuing throughout the experiment (i.e., 100 min). The percentage of inhibition of thrombus formation was determined and the ID₅₀ (dose that produced 50% inhibition of thrombus formation) values were estimated by linear regression.

### Results

### Optimization of TF-Induced Platelet Aggregation:

A number of factors were evaluated for their potential effect on TF-induced platelet aggregation, including the TF, calcium, FVIIa and intrinsic coagulation activation. Concentration-dependent studies (using a range of 1-100 μM) revealed that 10 μM Innovin® provided an optimum concentration of TF for producing a platelet aggregation response that was sufficiently sensitive to antithrombotic agents. The adjustment of Ca²⁺ concentration in the PRP was a feature for the assay since blood was sampled into a Ca²⁺-lowering sodium citrate solution. The addition of Ca²⁺ to 5 mM was not sufficient, while 10 mM was excessive, for driving the TF-induced platelet aggregation. The optimum concentration of Ca²⁺ for the assay was 7.5 mM. In addition, to reduce the background levels of thrombin generation it was necessary to block the intrinsic coagulation activation. Thus, 50 μg/ml CTI was directly added for blood sample during collection. In contrast, no difference was found for the addition of vehicle, 2, 20, 200 and 2000 M FVIIa in the PRP.

### Differential Effects of Antiplatelet Agents on TF-Induced Platelet Aggregation in Human PRP:

A panel of representative tracing for the effect of FVIIa inhibitor 2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid on TF-induced platelet aggregation is shown in FIG. 2. Dose-dependent effect for the time delay of platelet aggregation was demonstrated with 75, 175, 286, 671 and 865 seconds for 0, 1.26, 4.0, 12.6 and 25.2 μM 2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid, respectively (FIGS. 2 and 3). Similarly, dose-dependent effects were observed for FXa inhibitors 1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide (FIG. 3) and 1-(2-aminomethylphenyl)-3-trifluoromethyl-N-[3-fluoro-2′-(aminosulfonyl)[1,1′-biphenyl)]-4-yl]-1H-pyrazole-5-carboxyamide (data not shown), thrombin inhibitor argatroban (FIG. 3) and heparin (FIG. 3). No effect was observed for FXIa inhibitor 3′-((2S,4R)-6-Carbamimidoyl-4-methyl-4-phenyl-1,2,3,4-tetrahydro-quinolin-2-yl)-4-carbamoyl-5′-(3-methyl-butyrylamino)-biphenyl-2-carboxylic acid unless a very high dose (40 μM) was used (FIG. 3), which might already trigger a weak and non-selective inhibition of FVIIa. In contrast antiplatelet agents including the selective P2Y1 antagonists 1-[2-(2-tert-Butyl-phenoxy)-pyridin-3-yl]-3-(4-trifluoromethoxy-phenyl)-urea (FIG. 3) and N6-methyl-2′-deoxyadenosine-3′,5′-bisphosphate, and the selective P2Y12 antagonist cangrelor (data not shown), had no effect on the time delay of TF-induced platelet aggregation.

The 2×Base values were determined from the concentration-response study of each anticoagulant. The 2×Base values were 1.25, 4, 26 and 0.55 μM for FVIIa inhibitor (2′-((6R,6aR,11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid), FXa inhibitor (1-[3-(aminomethyl)phenyl]-N-[3-fluoro-2′-(methylsulfonyl)[1,1′-biphenyl]-4-yl]-3-(trifluoromethyl)-1H-pyrazole-5-carboxamide), FXIa inhibitor (3′-((2S,4R)-6-Carbamimidoyl-4-methyl-4-phenyl-1,2,3,4-tetrahydro-quinolin-2-yl)-4-carbamoyl-5′-(3-methyl-butyrylamino)-biphenyl-2-carboxylic acid) and thrombin inhibitor (argatroban), respectively. The 2×Base value for heparin was 0.2 units. The 2×Base values on the time delay of TF-induced platelet aggregation could not be determined for P2Y1 (1-[2-(2-tert-Butyl-phenoxy)-pyridin-3-yl]-3-(4-trifluoromethoxy-phenyl)-urea and N6-methyl-2′-deoxyadenosine-3′,5′-bisphosphate) and P2Y12 inhibitors since they had no effect on this specific read-out.

The ED₈₀ values are based on the effect of each compound to reach 80% inhibition of thrombus formation on a rabbit EACT model. The ED₈₀ values for FVIIa inhibitor (2′-((6R,6aR, 11bR)-2-Carbamimidoyl-5,6a,7,11b-tetrahydro-6H-indeno[2,1-c]quinolin-6-yl)-5′-hydroxy-4′-methoxy-biphenyl-4-carboxylic acid), FXa inhibitor (1-[3-(aminomethyl)phenyl]-N-[3-fluoro-2′-(methylsulfonyl)[1,1′biphenyl,]-4-yl]-3-(trifluoromethyl)-1H-pyrazole-5-carboxamide), FXIa inhibitor (3′-((2S,4R)-6-Carbamimidoyl-4-methyl-4-phenyl-1,2,3,4-tetrahydro-quinolin-2-yl)-4-carbamoyl-5′-(3-methyl-butyrylamino)-biphenyl-2-carboxylic acid) and thrombin inhibitor (argatroban) were 1.1, 0.4, 0.8, and 0.62 μM, respectively. The ED₈₀ value was not determined in the ECAT model for heparin. Instead, the IC₅₀ value for heparin was determined in a rabbit AV shunt model that was 20 units/kg/hr.

What is claimed is:

1. A method for differentiating compounds that modulate the extrinsic and/or intrinsic coagulation pathways, comprising:
a) collecting a blood sample in the presence of a calcium chelating agent and an inhibitor of contact activation,
b) collecting a first and second platelet-rich plasma (PRP) sample from the blood sample in (a),
c) adding a first test compound and a fibrin crosslinking inhibitor to said first PRP sample,
d) adding a second test compound and a fibrin crosslinking inhibitor to said second PRP sample,
e) incubating the mixtures from (c) and (d),
f) inducing platelet aggregation in the mixtures from (c) and (d),
g) measuring the time delay for said platelet aggregation in the mixtures from (c) and (d), and
h) differentiating said first and second test compounds by comparing the time delay for platelet aggregation measured in (g) for said first and second PRP samples.

2. A method for identifying a compound that modulates the extrinsic coagulation pathway, comprising:
a) collecting a blood sample in the presence of a calcium chelating agent and an inhibitor of contact activation,
b) collecting a PRP sample from the blood sample in (a),
c) adding a test compound and a fibrin crosslinking inhibitor to said PRP sample,
d) incubating the mixture from (c),
e) inducing platelet aggregation,
f) measuring the time delay for said platelet aggregation, and
g) identifying a compound that modulates the extrinsic coagulation pathway by comparing the time delay for platelet aggregation measured in (f) to the time delay for platelet aggregation measured in a control PRP sample to which no test compound is added.

3. A method for determining an effective dosage of an anticoagulant in a patient, comprising:
a) collecting a first blood sample from a patient in the presence of a calcium chelating agent and an inhibitor of contact activation,
b) collecting a first PRP sample from the blood sample in (a),
c) adding a fibrin crosslinking inhibitor to said first PRP sample,
d) administering an anticoagulant to said patient,
e) collecting a second blood sample from said patient in the presence of a calcium chelating agent and an inhibitor of contact activation,
f) collecting a second PRP sample from the blood sample in (e),
g) adding a fibrin crosslinking inhibitor to said second PRP sample,
h) inducing platelet aggregation in said first and second PRP samples,
i) measuring the time delay for platelet aggregation,
j) comparing the time delay for platelet aggregation in said first and second PRP samples, and
k) increasing or decreasing the dosage of said anticoagulant based on the results in (j).

4. The method of claim 1 wherein said fibrin crosslinking inhibitor is pefabloc.

5. The method of claim 1 wherein said PRP is human PRP.

6. The method of claim 1 wherein said incubating is for at least 3 min at about 37° C.

7. The method of claim 1 wherein said aggregation is induced by addition of tissue factor and Ca²⁺.

8. The method of claim 7, wherein the concentration of tissue factor is from about 1-100 μM.

9. The method of claim 7, wherein the concentration of tissue factor is about 10 μM.

10. The method of claim 7 wherein Innovin® is added as the tissue factor component.

11. The method of claim 7, wherein said Ca²⁺ is CaCl₂.

12. The method of claim 7, wherein the concentration of Ca²⁺ is from about 5-10 mM.

13. The method of claim 12, wherein the concentration of Ca²⁺ is about 7.5 mM.

14. The method of claim 1 wherein said inhibitor of contact activation is Corn Trypsin Inhibitor (CTI).

15. The method of claim 1 wherein said time delay is measured under continuous stirring at 1000 rpm.

16. The method of claim 3, wherein said anticoagulant is factor VIIa inhibitor.

17. The method of claim 1 wherein said calcium chelating agent is Na-Citrate.
